(19) United States
(12) Patent Application Publication
(10) Pub. No.: US 2009/0286307 A1
(43) Pub. Date: Nov. 19, 2009

(54) Automatic treatment system of leftover food treating device

(76) Inventors: Bok Gyeong Kim, Suwon-si (KR); Jin Hyun Shon, Seongnam-si (KR); Byeong Sik Choi, Seoul (KR)

Correspondence Address: MCDERMOTT WILL & EMERY LLP, 600 13TH STREET, N.W., WASHINGTON, DC, 20005-3096, US

(21) Appl. No.: 12/153,193

(22) Filed: May 15, 2008

(51) Int. Cl. C12M 1/36

(52) U.S. Cl. 435/286.5; 435/286.1

(57) ABSTRACT

The present invention relates to an automatic treatment system of a leftover food treating device comprising a setting means for receiving an output voltage of a voltage supplying means and setting control signals of the leftover food treating device; a controlling means for receiving input data through the setting means and controlling the operational state of the leftover food treating device; a sensing means for inputting measured data to the controlling means; a driving means for receiving the output signals of the controlling means; a displaying means for displaying the operational states of the setting means, the sensing means, the driving means, and the controlling means; and an alarming means for displaying an alarm setting value through the displaying means and outputting a predetermined alarm sound, in the case of the alarm setting value of a program preset to the controlling means.

### BACKGROUND OF THE INVENTION

1. Field of the Invention

The present invention relates to an automatic treatment system of a leftover food treating device, and more particularly to an automatic treatment system of a leftover food treating device in which collected food wastes (hereinafter, referred to as leftover food) can be treated cleanly and without bed smells by repeatedly and automatically performing the process of heating and agitating the leftover food and decomposing the leftover food with microorganisms.

2. Description of the Prior Art

Generally, leftover food discharged from homes cannot be treated together with general wastes and should be collected in separate plastic bags. However, since it is difficult to collect a small amount of leftover food discharged from homes in a plastic bag, it is occasionally thrown away together with general wastes. Further, even in the case in which the leftover food is collected in a plastic bag, since it rapidly goes rotten and generates bed smells severely, the air in home is contaminated and the treatment cost is excessive, thereby causing an economic burden.

Currently, as food service industry expands, homes or restaurants generate a large amount of leftover food everyday. Some of the leftover food is used as feeds of domestic animals, but due to the difficulty in carrying and treating the leftover food, almost all the leftover food is reclaimed or incinerated.

However, in the former reclamation method, bad smells are generated by nitrogen and sulfur compounds. Further, the atmosphere, the water, the soil, and the underground water are seriously contaminated by the high density leachate.

On the other hand, the latter incineration method requires incinerators of high capacities, contaminates the atmospheric environment due to harmful substances discharged into the air during the incineration. Further, the incinerating efficiency of leftover food is lowered by the low-caloric leftover food itself and by the moisture in the leftover food. Therefore, the incineration method is so uneconomical that it costs a lot of expenses to treat the leftover food. Further, harmful substances such as dioxin can be discharged from the leftover food due to the incomplete combustion of the leftover food.

Therefore, recently, various leftover food treating devices for treating leftover food by mechanical methods have been suggested, but have a problem in that they cause frequent disorders due to their unstable systems.

For example, leftover food is pressed to remove the moisture and is shattered to pieces. Thereafter, the moisture is discharged through a drain and the shattered pieces are collected to be used as composts or are incinerated. However, according to the leftover food treating device, since the extracted moisture is discharged through a drain, it contaminates a river. Further, it is bothersome to treat the shattered pieces after collecting them. Furthermore, errors are occasionally generated when the leftover food treating device is driven.

### SUMMARY OF THE INVENTION

Accordingly, the present invention has been made to solve the above-mentioned problems occurring in the prior art, and an object of the present invention is to provide an automatic treatment system of a leftover food treating device in which the microorganisms introduced into an agitation tank are activated by properly regulating oxygen and humidity, so that the leftover food can be efficiently treated, and which can be automatically operated stably and without disorder.

It is another object of the present invention to provide an automatic treatment system of a leftover food treating device in which leftover food is discharged after decomposed to water and carbon dioxide, thereby preventing the environmental contamination.

In order to accomplish this object, according to a preferred embodiment of the present invention, there is provided an automatic treatment system of a leftover treating device which comprises a body with which a door for opening and closing an introduction opening into which leftover food is introduced is engaged on the upper surface thereof, an agitation tank installed in the interior of the body and receiving the leftover food, with microorganisms introduced into the agitation tank, an agitation shaft transversely engaged with a central portion of the agitation tank, for agitating the leftover food, an voltage supplying means provided in a control panel on one side of the body, for supplying voltages to driving sections, the automatic treatment system comprising: a setting means for receiving an output voltage of the voltage supplying means and setting control signals of the leftover food treating device; a controlling means for receiving input data through the setting means and controlling the operational state of the leftover food treating device; a sensing means installed in the body, for inputting measured data to the controlling means; a driving means for receiving the output signals of the controlling means, which is obtained by comparing and calculating the data input through the sensing means and the data of the setting means and for driving the leftover food treating device; a displaying means for displaying the operational states of the setting means, the sensing means, the driving means, and the controlling means; and an alarming means for displaying an alarm setting value through the displaying means and outputting a predetermined alarm sound, in the case of the alarm setting value of a program preset to the controlling means.

### BRIEF DESCRIPTION OF THE DRAWINGS

The above and other objects, features and advantages of the present invention will be more apparent from the following detailed description taken in conjunction with the accompanying drawings, in which:

FIG. 1 is a perspective view for showing the appearance of a leftover food treating device including an automatic treatment system according to the present invention;

FIG. 2 is a first cross-sectional view for showing the leftover food treating device including the automatic treatment system according to the present invention, which is viewed from one side thereof;

FIG. 3 is a second cross-sectional view for showing the leftover food treating device including the automatic treatment system according to the present invention, which is viewed from the other side thereof;

FIG. 4 is a partially exploded perspective view for showing the leftover food treating device including an automatic treatment system according to the present invention;

FIG. 5 is a view for showing the constitution of a control panel of FIG. 1;

FIG. 6 is a block diagram for showing the automatic treatment system of the leftover food treating device according to the present invention; and

FIG. 7 is a flow chart for describing the control operation of the automatic treatment system of the leftover food treating device according to the present invention.

### DETAILED DESCRIPTION OF THE PREFERRED EMBODIMENTS

Hereinafter, a preferred embodiment of the present invention will be described in detail with reference to the accompanying drawings. It should be noted that the same elements in a drawing is endowed with the same reference numerals even though they are shown in the other drawings. Further, in the following description of the present invention, a detailed description of known functions and configurations incorporated herein is omitted to avoid making the subject matter of the present invention unclear.

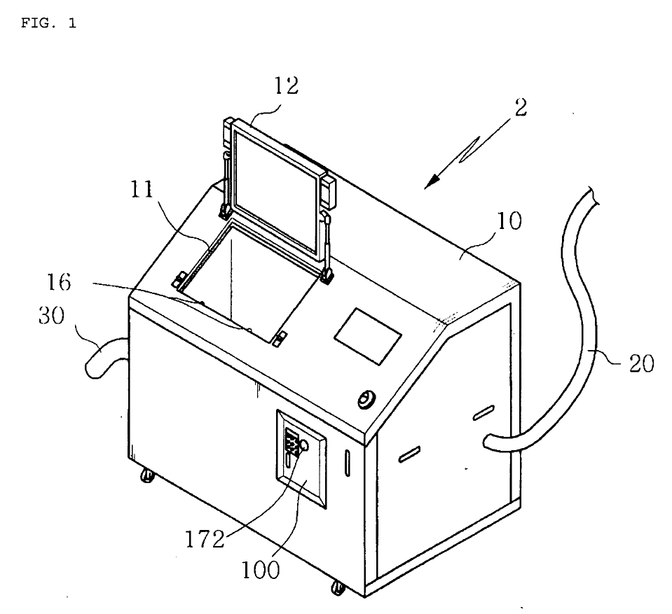

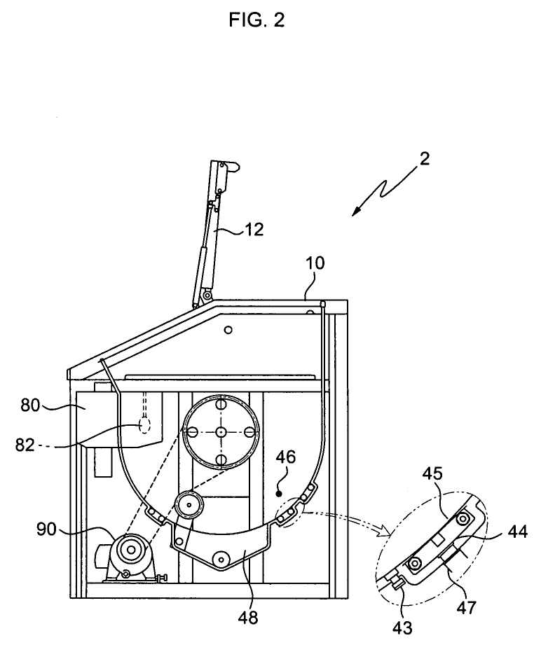

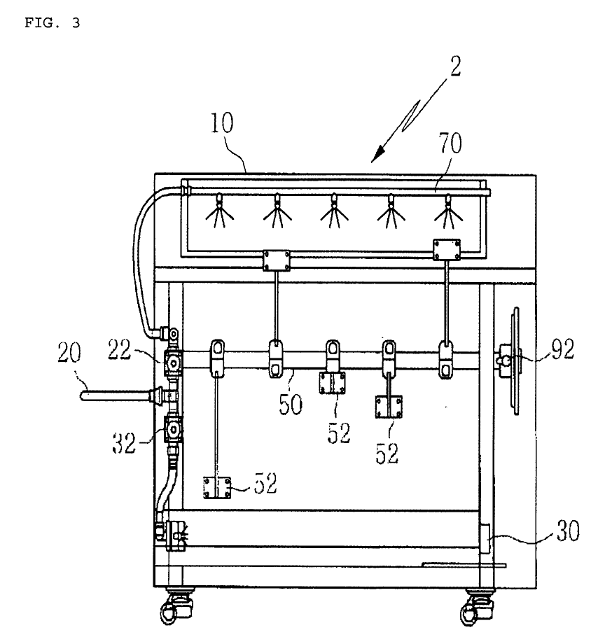

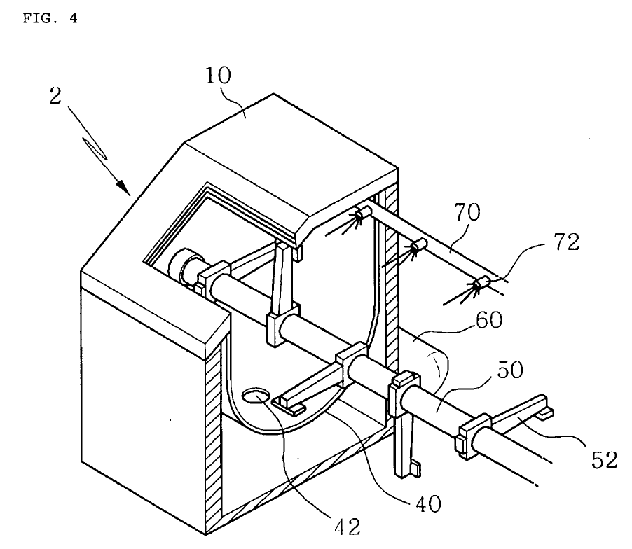

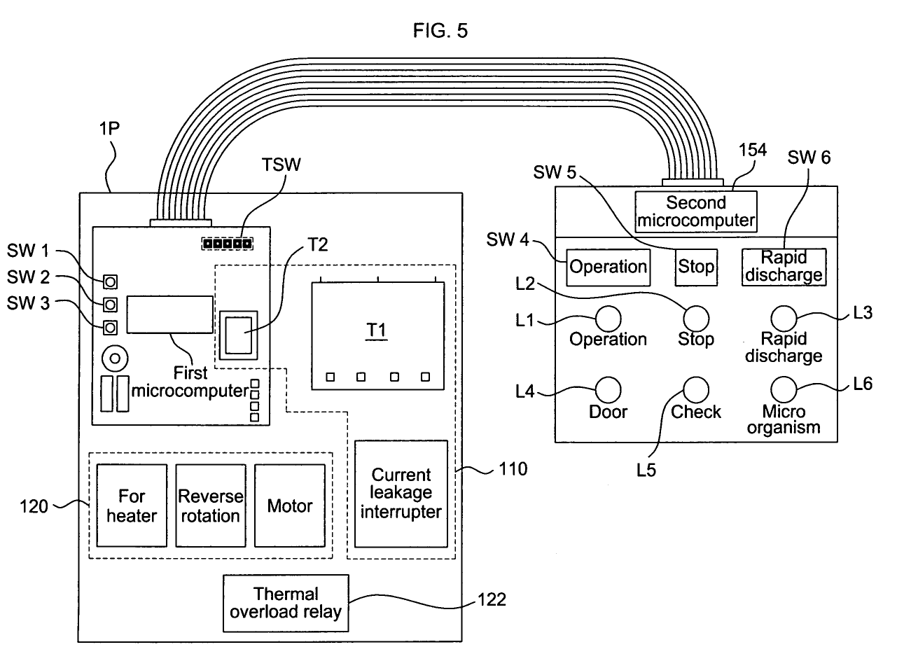

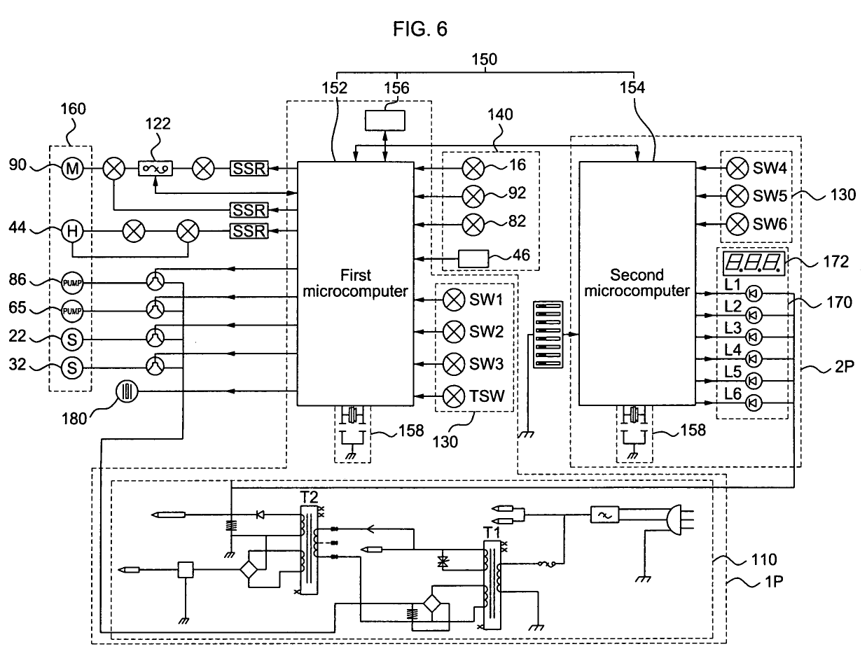

FIG. 1 is a perspective view for showing the appearance of a leftover food treating device including an automatic treatment system according to the present invention. FIG. 2 is a first cross-sectional view for showing the leftover food treating device including the automatic treatment system according to the present invention, which is viewed from one side thereof. FIG. 3 is a second cross-sectional view for showing the leftover food treating device including the automatic treatment system according to the present invention, which is viewed from the other side thereof. FIG. 4 is a partially exploded perspective view for showing the leftover food treating device including an automatic treatment system according to the present invention. FIG. 5 is a view for showing the constitution of a control panel of FIG. 1. FIG. 6 is a block diagram for showing the automatic treatment system of the leftover food treating device according to the present invention.

Referring to FIGS. 1 to 4, the leftover food treating device 2 according to the present invention includes a body 10 with which a door 12 for opening/closing an introduction opening 11 through which leftover food is introduced is engaged on the upper surface thereof, an agitation tank 40 installed in the interior of the body 10, into which the leftover food is introduced, with microorganisms introduced thereinto, an agitation shaft 50 transversely engaged with a central portion of the agitation tank 40 and including a plurality of agitation blades 52 for agitating the leftover food, and a control panel 100 provided on one side of the body 10 and including a voltage supplying means and a controlling means.

The body 10 includes a plurality of door switches for detecting the opened or closed state of the door 12. A spraying solenoid valve 22 for performing an opening/closing function of the water sprayed through a water supplying pipe 20 and transversely engaged at an upper end of the agitation tank 40 is provided on one side of the body 10. A discharging solenoid vale 32 for injecting water so that a discharge opening 30 connected to a water dropping portion 48 is not blocked is provided on the other side of the body 10.

A deodorizing microorganism tank 80 is provided in the interior of the body 10, and a float switch 82 for detecting the level is installed in the interior of the deodorizing microorganism tank 80.

A heating means for providing the agitation tank 40 with a suitable temperature and efficiently processing leftover food is installed on the lower side of the agitation tank 40. The heating means includes a heater 44 located on the right lower side of the agitation tank 40, a contact switch 47 installed in the heater 44, for switching the heater 44 on and off if the temperature of the heater 44 reaches a predetermined temperature, a heat conducting plate 45 surrounding the lower side surface of the agitation tank 40 for transferring the heat generated from the heater 44 to the agitation tank 40, and a temperature sensor 46 for measuring the temperature in the heated agitation tank 40. The aerobic microorganisms in the agitation tank 40 are actively proliferated by the transferred heat.

Further, an oxygen supplying means is provided on one side of the agitation tank 40. The oxygen supplying means includes an air compressor 60 mounted to the rear side of the body 10 and an oxygen supplying opening 43 provided on one side of the agitation tank 40, for supplying the oxygen supplied by the operation of the air compressor 60 into the agitation tank 40. Therefore, the oxygen supplied into the agitation tank 40 optimizes the activation of the microorganisms.

In FIG. 5, the control panel 100 is provided on one side of the leftover food treating device in order to function as a controlling section. A first PCB 1P is provided in the interior of the control panel 100, and a second PCB 2P is provided at the door of the control panel 100. The first and second PCBs 1P and 2P electrically transfer signals (data) mutually.

The first PCB 1P includes a voltage supplying means for receiving a predetermined AC voltage and converting and lowering it to a predetermined DC voltage to supply it, and a thermal overload relay 122 for detecting overload states of an AC electric contactor 120 (for a heater, for driving a motor, and for reversely rotating a motor) used in drive sections after a voltage is supplied from the voltage supplying means and the drive motor 90. The first PCB 1P comprises a first switch SW1 for setting the agitation time, a second switch SW2 for setting the spraying time, and a third switch SW3 for setting the temperature, for setting data. Further, the first PCB 1P comprises a motor, a heater, a solenoid valve, and a test switch TSW for driving a pump and selecting an automatic mode, and comprises a first microcomputer 152 for controlling the elements on the whole.

On the other hand, the second PCB 2P is provided on the rear surface of the door of the control panel 100. The second PCB 2P includes a fourth switch SW4 for starting the operation, a fifth switch for selecting a stop operation, and a sixth switch SW6 for selecting a rapid discharge. A plurality of lamps are disposed under the fourth to sixth switches SW4 to SW6. The second PCB 2P comprises a first lamp L1 for representing the operation state, a second lamp L2 for representing the voltage supplying state, a third lamp L3 for representing the rapid discharge, a fourth lamp L4 for representing whether the door 11 is opened or closed, a fifth lamp L5 for representing whether the device is in a check mode, and a sixth lamp L6 for representing whether the level of the microorganisms is a low level. Further, the second PCB 2P comprises a second microcomputer M2 to which a control signal of the first microcomputer M1 is applied to driving the operation thereof. The second PCB 2P further comprises a display section 172 (refer to FIG. 1) for representing the operational state. Further, a buzzer (not shown) for generating an alarm sound is provided in the second PCB 2P.

Referring to FIG. 6, the automatic treatment system of a leftover treating device a voltage supplying means 110 for supplying voltages to driving sections, a setting means 130 for receiving an output voltage of the voltage supplying means and setting control signals of the leftover food treating device 2, a controlling means 150 for receiving input data through the setting means 130 and controlling the operational state of the leftover food treating device 2, a sensing means 140 installed in the body 10, for inputting measured data to the controlling means 150, a driving means 160 for receiving the output signals of the controlling means 150, which is obtained by comparing and calculating the data input through the sensing means 140 and the data of the setting means 130 and for driving the leftover food treating device, a displaying means 170 for displaying the operational states of the setting means 130, the sensing means 140, the driving means 160, and the controlling means 150, and an alarming means 180 for displaying an alarm setting value through the displaying means 170 and outputting a predetermined alarm sound, in the case of the alarm setting value of a program preset to the controlling means.

The voltage supplying means 110 comprises a current leakage interrupter 112 for detecting the current leakage of an input AC voltage source 220V, a fuse 114 provided at the output end of the current leakage interrupter 112, for protecting a circuit by interrupting the voltage supply when a current and a voltage are formed in the voltage source, and a first transformer T1 and a second transformer T2 for lowering the voltage input through the fuse 114 to a predetermined voltage, and a static voltage IC for outputting the voltage lowered by the first and second transformer T1 and T2 stably and constantly.

The setting means 130 comprises a first switch SW1 provided on one side of a first PCB 1P in the control panel 100, for setting an agitation time of the agitation shaft 50 (refer to FIG. 3), a second switch SW2 provided one side of the first switch SW1, for setting a sprinkling time, a third switch SW3 provided on one side of the second switch SW2, for setting a temperature, a test switch TSW provided on the other side of the first PCB 1P, for testing drive sections of the leftover food treating device, a fourth switch SW4 electrically connected to the first PCB 1P and provided in the second PCB 2P disposed in the control panel 100, for starting the operation of the leftover food treating device after the mode is converted (to the test mode) through the test switch TSW, a fifth switch SW5 for stopping the operation of the leftover food treating device after the leftover food treating device has been operated through the fourth switch SW4, and a sixth switch SW6 for stopping the treatment of the leftover food treating device operated through the fourth switch SW4 and for rapidly discharging the leftover food in the agitation tank 30 (refer to FIG. 3). On the other hand, as mentioned above, the fourth and fifth switches SW4 and SW5 are selected to perform the test after the mode is converted to the test mode, and the leftover food treating device according to the present invention is automatically operated without turning on the fourth switch SW4 whenever leftover food is introduced.

The controlling means 150 comprises a first microcomputer 152 provided in the first PCB 1P, for generally controlling the leftover food treating device, a second microcomputer 154 for a sub-controller which is provided in the second PCB 2P, for receiving output signals from the first microcomputer 152 and controlling the displaying means 170, and a memory 156 for storing the data of the first microcomputer 152.

The displaying means 170 comprises a first lamp L1 disposed in a door of the control panel 100, for receiving output signals from the second microcomputer 154 and displaying the operational state of the leftover food treating device, a second lamp L2 disposed on one side of the first lamp L1, for displaying whether a voltage to the leftover food treating device is supplied or interrupted, a third lamp L3 disposed on one side of the second lamp L2, for displaying a rapid discharge state of the treated water which is decomposed, a fourth lamp L4 disposed at the lower end of the first lamp, for displaying an opened state of the door opening and closing the introduction opening 11 by flickering if the door 12 is opened, a fifth lamp L5 disposed at the lower end of the second lamp L2, for flicking if an abnormal signal is generated during the normal operation of the leftover food treating device, and a sixth lamp L6 disposed at the lower end of the third lamp L3, for flickering if the level of the microorganisms supplied into the agitation tank is determined to be a low level.

The driving means 160 comprises a drive motor 90 installed on one side of the body 10 of the leftover food treating device, for rotating the agitation shaft 90, a heater 44 installed on the right lower side of the agitation tank 40, for raising the temperature in the agitation tank 40, a microorganism supplying pump 86 for performing pumping so as to supply microorganisms into a microorganism tank 80 installed on one side of the interior of the body 10, an air compressor installed on the rear side of the body 10, a spraying solenoid valve 22 disposed on the upper side of the interior of the agitation tank 40, the spraying solenoid valve being opened or closed by an output signal of the controlling means 150 so that water is supplied to an injection nozzle 72 for spraying water, and a discharging solenoid valve 32 provided on one side of the agitation tank 40, for injecting water so that a discharge opening 30 connected to a water dropping portion 48 cannot be blocked after the decomposed substances generated in the agitation tank 40 has dropped to the water dropping portion 48.

The sensing means 140 comprises a temperature sensor 46 for detecting the temperature in the interior of the agitation tank 40 heated by the heater 44, a contact switch 47 installed in the heater 44, for switching the heater 44 on or off if the heater 44 reaches a predetermined temperature, a door switch 16 installed at an upper portion of the body 10, for detecting the opened or closed state of the door 12, a rotation detecting sensor 92 for detecting the rotational state of the drive motor 90, and a float switch 82 provided in the interior of the microorganism tank 86, for detecting the level of the microorganisms supplied through the microorganism supplying pump 86.

The alarming means 180 comprising a buzzer for outputting a predetermined alarm sound in the case in which a predetermined value of a program preset to the controlling means is generated during the normal operation of the leftover food treating device.

The leftover food treating device further comprises an overload detecting means including a thermal overload relay for detecting an overload state if an overload is generated in the drive motor 90 and applying an electric signal to the controlling means 150.

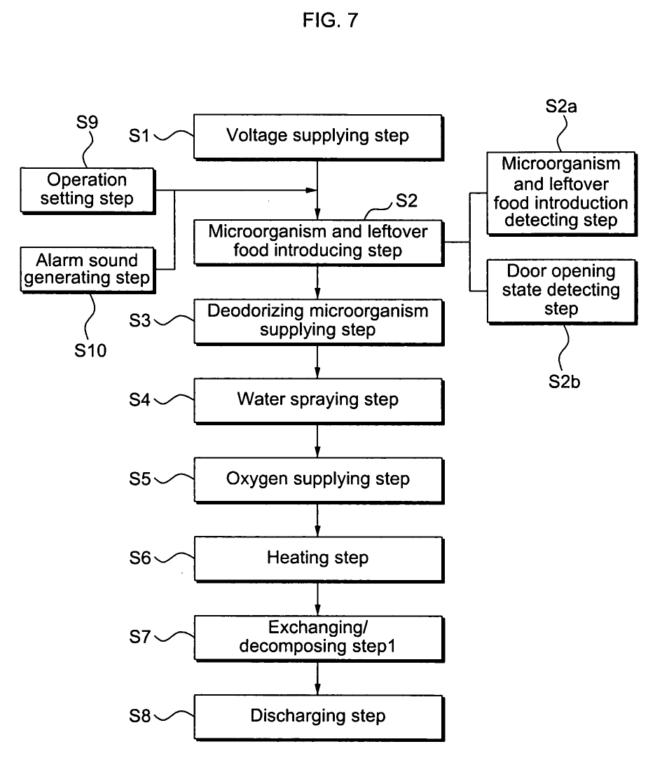

Hereinafter, the control operation of the automatic treatment system of the leftover food treating device according to the present invention will be described with reference to FIGS. 6 and 7.

The control operation of the automatic treatment system of the leftover food treating device according to the present invention comprises a voltage supplying step S1 in which an operation waiting state is maintained by applying a drive voltage through the voltage supplying means 110, a microorganism and leftover food introducing step S2 in which leftover food is introduced in the state in which biochips (which is introduced into the agitation tank so that the microorganisms can be mixed therewith to live thereupon and includes sawdust) and the microorganisms are introduced into the agitation tank 40 by opening the door 12 after the leftover food treating device becomes in the operation waiting state and then the door 12 is closed, a deodorizing microorganism supplying step S3 in which deodorizing microorganisms are supplied into the microorganism tank 80 having a predetermined size, in which the float switch 82 is installed, after the microorganism and leftover food introducing step S2, a spraying step S4 in which a predetermined amount of water is injected to provide a humidity suitable for the microorganisms through the injecting nozzles 72 installed at the upper end of the agitation tank 40 after the odorizing microorganism supplying step S3, an oxygen supplying step S5 in which oxygen is supplied into the agitation tank 40 to activate the microorganisms after the spraying step, a heating step S6 in which the aerobic microorganisms in the agitation tank 40 are proliferated by heating the agitation tank 40 with the heater 44 after the oxygen supplying step S5, an agitating/decomposing step S7 in which the leftover food is agitated and decomposed by driving the drive motor 90 at a predetermined interval after the heating step S6, and a discharging step S8 in which the liquefied fluid (decomposed substances), which is generating by decomposing the microorganisms is discharged if the leftover treatment is completed through the agitating/decomposing step S7.

Hereinafter, the control operation performed in each step will be described in detail.

First, in the voltage supplying step S1, the mode is converted to a power-saving mode and only the maximum operation is performed, if the door is not opened or closed for twenty four hours.

Further, the microorganism and leftover food introducing step S2 comprises a microorganism and leftover food introduction detecting sub-step S2a in which it is determined that the leftover food is not introduced if the opening time for introducing the leftover food is within a predetermined range (for example, the opening time of the door is within 10 seconds or the interval from the former opening time is within 5 minutes) and an alarm sound is output through the buzzer of the alarming means 180 if the opening time of the door is more than a predetermined time (for example, the door is opened for more than 5 minutes) after the voltage supplying step S1 and a door opened state detecting sub-step S2b in which all operation of the leftover food treating device is temporarily stopped if the opening state of the door is detected by the door sensor 16 while the leftover food treating device is being normally operated through the microorganism and leftover food detecting sub-step S2a.

Further, the deodorizing microorganism supplying step S3 performs a function for injecting a predetermined amount of microorganisms and then reducing bed smells when the door 12 is opened. In the preferred embodiment of the present invention, the microorganisms are injected (by approximately 5 cc) every two hour to decompose the bed smells generated during the operation of the leftover food treating device by using the deodorizing microorganisms.

Further, in the spraying step S4, water is sprayed after the door 12 is opened to introduce leftover food through the initial introduction opening 11 and then is closed. In the preferred embodiment of the present invention, the water is sprayed every one hour during the operation of the leftover food treating device. The spraying step S4 functions as a lubricating operation for settling the overload before the drive motor 90 is reversely rotated if the overload is detected.

Further, in the oxygen supplying step S5, the microorganisms are actively proliferated by supplying a predetermined amount of oxygen when the drive motor 90 is stopped. Especially, in the preferred embodiment of the present invention, the oxygen is supplied for one minute at a time interval of 10 minutes.

Further, the heating step S6 is a step for maintaining the set temperature of the heater 44 at a predetermined temperature (for example, 85 degrees Celsius), with the heater 44 not being influenced by the change of the surrounding temperature. If the temperature of the heater 44 is more than a predetermined temperature, the contact switch 47 is turned off, and if the temperature of the heater 44 is less than a predetermined temperature, the contact switch 47 is turned on. Therefore, the leftover food is prevented from being stuck to the agitation tank 40.

Further, the agitating/decomposing step S7 is performed to agitate and decompose the leftover food by driving the drive motor 90 at a predetermined interval. Especially, in the preferred embodiment of the present invention, the agitating operation is performed for one minute every 10 minute of normal operation. If the overload is detected in the overload detecting means 122 in such a case in which the agitation blades 52 are caught by foreign substances, the agitation shaft is reversely rotated. This process is performed several times.

Further, the discharging step S8 is a step for discharging the decomposed substance, which has been decomposed and liquefied by the microorganisms, through a discharging opening by opening the discharging solenoid, after the agitating and decomposing step S7.

On the other hand, an operation setting step S9 can be provided between the voltage supplying step S1 and the leftover food introducing step S2, and the operation setting process (selected from a plurality of data) is as follows.

SW1 (Setting the agitation time): The agitation time 3/6/9/12 minutes during the normal operation

SW2 (Setting the amount of sprayed water during the agitation): The spraying time 5/10/20/30 minutes during the normal operation

SW3 (Setting the temperature of the agitation tank during the operation): The temperature range during the normal operation 0/30/35/40/45/50/55/60 degrees Celsius. It is preferable that the temperature of the agitation tank is set to more than 40 degrees Celsius basically.

TSW (Selecting the test mode of the leftover food treating device)

SW4 (Operating (driving) the leftover food treating device)

SW5 (Stopping the operation of the leftover food treating device)

SW6 (Rapidly discharge in the case of decomposition delay due to excessive introduction of the leftover food during the operation)

Further, an alarm sound generating step S10 for generating an alarm sound through the buzzer of the alarming means 180 if an abnormal signal of the leftover food treating device is detected can be provided between the voltage supplying step S1 and the leftover food introducing step S2.

The alarm sound is generated in a case in which the operational switches are manipulated in the state in which the door 12 is opened after the voltage is supplied, in a case in which five minutes have passed after the door 12 was opened, in a case in which an over-current is detected in the drive motor 90, in a case in which the level of the odorizing microorganisms is low, and in the case of the alarm mode.

Further, a rapid discharge step S11 in which the leftover food is rapidly discharged by turning the rapid discharge selecting switch SW6 in the case of decomposition delay due to the excessive introduction of the leftover food is provided between the agitating/decomposing step S7 and the discharging step S8.

Further, an operational state displaying step S12 in which the operational states of the voltage supplying step S1 to the discharging step S8 are displayed through the lamps provided in the control panel 100 by the control signals of the controlling means 150 can be further performed. In this case, the operational states are displayed through the first to sixth lamps L1 to L6 provided in the control panel 100.

Finally, an operation testing step S13 in which the operational states of the drive sections of the leftover food treating device can be confirmed by converting the mode to a test mode after the voltage supplying step S1 can be further performed.

Hereinafter, the operation and effect of the automatic treatment system of the leftover food treating device according to the present invention will be described in detail with reference to FIGS. 1 to 6.

First, microorganisms capable of fermenting leftover food most ideally when the temperature, the humidity, and the oxygen are properly maintained are selected and are introduced in to the agitation tank 40. The microorganisms for decomposing the leftover food are those which do not harm to human bodies, and includes sixty kinds of different ferment bacilli and enzymes which can facilitate the digestion and decomposition of the fibroid material, the protein, the fat, and the carbohydrate.

In the state in which the microorganisms are introduced into the agitation tank 40, after a suitable amount of leftover food is introduced into the agitation tank 40, the interior of the body 10 is closed by closing the door 12.

Thereafter, if a voltage is applied through the setting means 130 of the control panel 100 provided on the front surface of the body 10, oxygen is introduced into the agitation tank 40 via the oxygen supplying pipe 64 of the agitation tank 40 so that the microorganisms can be actively proliferated. Further, water is sprayed through the plurality of injection nozzles 72 to obtain the optimal humidity for activating the microorganisms.

Further, if a voltage is applied to the drive motor 90, the agitation shaft 50 is rotated.

If the agitation shaft 50 connected to the drive motor 90 is rotated in one direction for a predetermined period of time and the agitation blades agitate and decompose the leftover food mixed with the microorganisms, the leftover food is changed to water and carbon dioxide by biochemical reactions. The water generated by the biochemical reactions and the moisture contained in the leftover food is discharged through the discharging opening 30.

Namely, if the leftover food is agitated and decomposed for a predetermined time, the leftover food agitated in the agitation tank 40 is changed to water and carbon dioxide and the moisture is dropped through the discharging portion 121.

As mentioned above, the leftover food is decomposed to water and carbon dioxide by the microorganisms, and the decomposed leftover food does not harm to the atmosphere, the water, and the soil. Therefore, the leftover food can be treated efficiently and environment-friendly.

The continual operations can be continuously performed for a predetermined time period according to the characteristics of the microorganisms used as a fermenting material and can be performed by introducing a predetermined amount of leftover food, the oxygen, and the water at every predetermined time according to the treating capacity of the device. Further, the leftover food is automatically treated and an operator can easily confirm the operational state by displaying the operational state through the displaying means. Further, if an abnormal signal of the leftover food treating device is detected, an operator is informed of an alarm sound through the buzzer.

As mentioned above, according to the present invention, the leftover food is decomposed to water and carbon dioxide, and the decomposed leftover food does not harm to the atmosphere, the water, and the soil. Therefore, the leftover food can be treated efficiently and environment-friendly.

Further, according to the present invention, the positive and reverse rotations of the drive motor can be selectively performed, and the agitation of the leftover food can be performed without the excess of the driving means, and the overload can be prevented in advance through the overload detecting means.

Further, according to the present invention, the operational state of the leftover food treating device can be confirmed through the displaying means, and even in the case in which an abnormal signal is detected, an operator can promptly cope with the situation by outputting an alarm sound.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. An automatic treatment system of a leftover treating device which comprises a body with which a door for opening and closing an introduction opening into which leftover food is introduced is engaged on the upper surface thereof, an agitation tank installed in the interior of the body and receiving the leftover food, with microorganisms introduced into the agitation tank, an agitation shaft transversely engaged with a central portion of the agitation tank, for agitating the leftover food, an voltage supplying means provided in a control panel on one side of the body, for supplying voltages to driving sections, the automatic treatment system comprising:
a setting means for receiving an output voltage of the voltage supplying means and setting control signals of the leftover food treating device;
a controlling means for receiving input data through the setting means and controlling the operational state of the leftover food treating device;
a sensing means installed in the body, for inputting measured data to the controlling means;
a driving means for receiving the output signals of the controlling means, which is obtained by comparing and calculating the data input through the sensing means and the data of the setting means and for driving the leftover food treating device;
a displaying means for displaying the operational states of the setting means, the sensing means, the driving means, and the controlling means; and
an alarming means for displaying an alarm setting value through the displaying means and outputting a predetermined alarm sound, in the case of the alarm setting value of a program preset to the controlling means.

2. An automatic treatment system according to claim 1, wherein the setting means comprises:
a first switch provided on one side of a first PCB in the control panel, for setting an agitation time of the agitation shaft;
a second switch provided one side of the first switch, for setting a sprinkling time;
a third switch provided on one side of the second switch, for setting a temperature;
a test switch provided on the other side of the first PCB, for testing drive sections of the leftover food treating device;
a fourth switch electrically connected to the first PCB and provided in the second PCB disposed in the control panel, for starting the operation of the leftover food treating device after the mode is converted through the test switch;
a fifth switch for stopping the operation of the leftover food treating device after the leftover food treating device has been operated through the fourth switch; and
a sixth switch for stopping the treatment of the leftover food treating device operated through the fourth switch and for rapidly discharging the leftover food in the agitation tank.

3. An automatic treatment system according to claim 1 or 2, wherein the controlling means comprises:
a first microcomputer provided in the first PCB, for generally controlling the leftover food treating device;
a second microcomputer provided in the second PCB, for receiving output signals from the first microcomputer and controlling the displaying means; and
a memory for storing the data of the first microcomputer.

4. An automatic treatment system according to claim 1 or 2, wherein the displaying means comprises:
a first lamp disposed in a door of the control panel, for receiving output signals from the second microcomputer and displaying the operational state of the leftover food treating device;
a second lamp disposed on one side of the first lamp, for displaying whether a voltage to the leftover food treating device is supplied or interrupted;
a third lamp disposed on one side of the second lamp, for displaying a rapid discharge state of treated water which is decomposed;
a fourth lamp disposed at the lower end of the first lamp, for displaying an opened state of the door by flickering if the door is opened;
a fifth lamp disposed at the lower end of the second lamp, for flicking if an abnormal signal is generated during the normal operation of the leftover food treating device;
a sixth lamp disposed at the lower end of the third lamp, for flickering if the level of the microorganisms supplied into the agitation tank is determined to be a low level; and
a display section for displaying the operational temperature of the leftover food treating device.

5. An automatic treatment system according to claim 1, wherein the driving means comprises:
a drive motor installed on one side of the body, for rotating the agitation shaft;
a heater installed on one side of the agitation tank, for raising the temperature in the agitation tank;
a microorganism supplying pump for performing pumping so as to supply microorganisms into a microorganism tank installed on one side of the interior of the body;
a spraying solenoid valve disposed on the upper side of the interior of the agitation tank, the spraying solenoid valve being opened or closed by an output signal of the controlling means so that water is supplied to an injection nozzle for spraying water; and
a discharging solenoid valve provided on one side of the agitation tank, the discharging solenoid valve being opened or closed by an output signal of the controlling means to inject water so that a discharge opening connected to a water dropping portion cannot be blocked after the decomposed substances generated in the agitation tank has dropped to the water dropping portion.

6. An automatic treatment system according to claim 1 or 5, wherein the sensing means comprises:
a contact switch installed in the heater, for switching the heater on or off if the heater reaches a predetermined temperature;
a temperature sensor for detecting the temperature in the interior of the agitation tank heated by the heater;
a door switch installed at an upper portion of the body, for detecting the opened or closed state of the door;
a rotation detecting sensor for detecting the rotational state of the drive motor; and
a float switch provided in the interior of the microorganism tank, for detecting the level of the microorganisms supplied through the microorganism supplying pump.

7. An automatic treatment system according to claim 1, wherein the alarming means is a buzzer for outputting a predetermined alarm sound in the case in which a predetermined value of a program preset to the controlling means is generated during the normal operation of the leftover food treating device.

8. An automatic treatment system according to claim 1, wherein the leftover food treating device further comprises an overload detecting means for detecting an overload state if an overload is generated in the drive motor and applying an electric signal to the controlling means.
